(19) United States
(12) Patent Application Publication
(10) Pub. No.: US 2006/0216321 A1
(43) Pub. Date: Sep. 28, 2006

(54) Solvent based processing technologies for making tissue/polymer composites

(75) Inventors: Suping Lyu, Maple Grove, MN (US); Jeff Gross, Memphis, TN (US); Sean Haddock, Memphis, TN (US); Chris Hobot, Tonka Bay, MN (US); Matt Kinnane, Bartlett, TN (US); Brian Loy, Bloomington, IL (US); Jim Schley, Coon Rapids, MN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SDGI HOLDINGS, INC.

(21) Appl. No.: 11/088,957

(22) Filed: Mar. 24, 2005

(51) Int. Cl. A61K 9/00

(52) U.S. Cl. 424/422; 424/400

(57) ABSTRACT

An improved osteoimplant device and method for producing it. Bone particles are mixed with a polymer and a solvent. The mixture is formed into the shape of an osteoimplant and substantially all of the solvent is removed. The osteoimplant may be osteoinductive, osteoconductive, and load-bearing.

### FIELD OF THE INVENTION

The present invention relates generally to bone-polymer osteoimplants and more specifically to bone-polymer osteoimplants manufactured with a solvent to facilitate polymer chain entanglement for better mechanical integrity.

### BACKGROUND

Osteoimplants are commonly used to replace diseased or damaged bone, to stabilize fractures, promote healing, promote bone fusion, and separate other bony elements. The use of autogenic (i.e. patient is the source), allogenic (i.e. another person is the source), and xenogenic (i.e. an animal is the source) bone material in composite osteoimplants is known. Inclusion of bone in the osteoimplant may provide structural reinforcement and impart osteoinductive properties (i.e. promotes bone growth) because of the presence in the bone of bone morphogenic protein and possibly other biologically advantageous compounds and nutrients. While in some cases entire implants may be produced by machining or compressing larger bones to smaller sizes, it also is common that bone material be ground, combined with other advantageous materials, and then shaped to a given specification by pressing, molding, machining, or otherwise. For example, osteoimplants may be manufactured by grinding autogenic, allogenic or xenogenic bone into small particles, powder, granules, or chips and then mixing the bone material with synthetic or natural polymers.

The use of polymers in the osteoimplant may result in a device that is osteoconductive, or suitable as a template for new bone growth, because the polymer provides a rigid and integrated structure in which the new bone cells are supported while new bone growth proceeds through the osteoimplant. This is especially useful for the production of load-bearing osteoimplants where the polymer must perform the load-bearing function of the osteoimplant while new bone growth proceeds. Bone-polymer osteoimplants are commonly produced by mixing bone material with the appropriate polymer and molding the composite into the implant shape. The implant shaping step may be followed by freeze drying or some other process to further harden the bone-polymer osteoimplant in preparation for implantation. The bone material also may be subjected to cleaning or other chemical treatments prior to mixing with the polymer in order to attain various biological advantages. Additionally, agents such as porosity generation agents, additional nutrients, antibiotics, antiretroviral drugs, and other biologically advantageous compounds are sometimes added to the bone-polymer mixture or otherwise incorporated into the osteoimplant.

During manufacture of the bone-polymer osteoimplant, it is important that the polymer chains form a continuous matrix that encapsulates and holds the bone particles. Otherwise, the osteoimplant will not achieve maximum strength. A continuous intermolecular matrix of polymers is created-through polymer welding, or at the molecular level, chain entanglement, wherein long polymeric chains are intertwined into an intermolecular structure. To achieve polymer chain entanglement, the individual polymer chains are brought into a state wherein they may move about freely and interpenetrate within each other to reach equilibrium.

To promote polymer entanglement, polymers may be processed in melt state. Processing the bone-polymer mixture in melt state, however, poses some disadvantages. In order to provide structural rigidity, polymers appropriate for use in an implantable device typically must have a glass transition temperature or melting temperature higher than human body temperature. Therefore, the melt state temperature of the bone-polymer mixture necessary to promote polymer chain entanglement also will be greater than normal body temperature. Process temperatures in excess of body temperature, however, may damage the bioactivity of the incorporated bone particles.

Various aqueous solutions or wetting agents also may be employed to induce chain entanglement during processing of bone-polymer osteoimplants. Depending upon the solubility of the polymer in aqueous solution, bringing the polymer into solution allows the polymer macromolecules to move about freely and become intertwined. Unfortunately, many polymers are only slightly soluble or completely insoluble in water. Therefore, processing of bone-polymer osteoimplants in water typically produces a slurry that neither results in a contiguous intermolecular matrix nor maximizes the strength of the finished osteoimplant.

Glycol based solvents have been used to prepare osteoimplants comprising bone particles and carriers, such as polyhydroxy compounds. However, similar to water, glycol based solvents do not dissolve most polymers. Also, glycol is not easy to remove from osteoimplant products due to its high boiling point. Finally, the residual glycol found in the osteoimplant may cause biological problems because glycol in high concentrations is known to be a neurotoxin.

Mechanical mixing of the bone-polymer osteoimplant material also may be employed to encourage entanglement. Though mechanical mixing may effectively disperse the bone material in the polymer matrix, mechanical mixing, like aqueous solutions, only slightly, or not at all, helps to produce the degree of entanglement necessary to achieve maximum strength in the osteoimplant.

The description herein of problems and disadvantages of known apparatus, methods, and devices is not intended to limit the invention to the exclusion of these known entities. Indeed, embodiments of the invention may include one or more of the known apparatus, methods, and devices without suffering from the disadvantages and problems noted herein.

### SUMMARY

An improved method of manufacturing a bone-polymer osteoimplant would be advantageous. A number of advantages associated with the embodiments are readily evident to those skilled in the art, including economy of design and resources, ease of use, quality of final product, cost savings, etc.

It therefore is a feature of an embodiment of the present invention to provide a method of making a bone-polymer osteoimplant that provides mixing of the components and results in an implant having improved properties. In accordance with these and other features of various embodiments of the invention, there is provided a method for producing an osteoimplant. The method comprises mixing bone material with a polymer and a solvent, forming the mixture into a suitable shape for an osteoimplant, and removing substantially all of the solvent to produce an osteoimplant.

Another feature of an embodiment of the invention provides a device created by the foregoing method.

Still further features and advantages of the present invention are identified in the ensuing description.

### DETAILED DESCRIPTION

The following description is intended to convey a thorough understanding of the present invention by providing a number of specific embodiments and details involving production of a bone-polymer osteoimplant. It is understood, however, that the present invention is not limited to these specific embodiments and details, which are exemplary only. It is further understood that one possessing ordinary skill in the art, in light of known systems and methods, would appreciate the use of the invention for its intended purposes and benefits in any number of alternative embodiments.

In one exemplary embodiment, a method of fabricating a bone-polymer osteoimplant is disclosed. Bone material, a polymer, and a solvent are mixed together, agitated, and optionally molded into the shape of the desired osteoimplant. The solvent then is substantially removed from the osteoimplant, for example, by freeze-drying. Throughout this description, “substantially removing” the solvent denotes removing at least 90%, preferably at least 95%, more preferably at least 99%, and most preferably at least 99.99% of the solvent. Those skilled in the art will appreciate that a trace amount of solvent may still remain in the implant even after substantial removal of the solvent by evaporation, freeze drying, or another method.

The bone material utilized in the fabrication of the osteoimplant may be obtained from any appropriate source bone. For example, the bone material may be derived from nondemineralized source bone, demineralized source bone, or a combination thereof. Demineralized source bone refers to source bone wherein at least some portion of the inorganic mineral content of the bone has been removed. Typical bone is about 70% by weight inorganic minerals. The remaining 30% is a combination of collagen and non collagenous proteins, including bone morphogenic protein (BMP), which is thought to have osteoinductive properties. The advantageous osteoinductive effect of bone morphogenic protein is inactivated by the inorganic mineral matrix of the bone. Therefore, at least partial demineralization of the source bone may be a preferred treatment before inclusion of the bone in the bone-polymer osteoimplant.

The demineralization process typically involves contacting the bone with an acidic solution. The acidic solution may be either an inorganic or organic acid. For example, the acid solution may be a solution of sulfuric acid, hydrochloric acid, or acetic acid. The shape of the bone, acidity of the solution, and temporal duration of the demineralization process determines how much of the inorganic minerals are removed from the source bone. These variables may be adjusted to achieve a certain level of demineralization. Following acid treatment, the source bone may be rinsed with water, for example de-ionized water or sterile water, to remove any remaining acid solution.

Removal of all or some of the demineralized bone's inorganic contents weakens the bone, consequently reducing its reinforcement effect on the osteoimplants. Therefore, by mixing amounts of demineralized bone and nondemineralized bone, osteoimplants of varying strength may be obtained. For example, when an extremely strong bone-polymer osteoimplant is desired, it is preferred that the bone material be prepared from primarily nondemineralized or only slightly demineralized source bone. In another exemplary embodiment of the present invention, nondemineralized or only slightly demineralized bone material is concentrated in the area of the implant that will be under the greatest load upon implantation.

In a preferred embodiment of the present invention, the demineralization of the source bone is preceded by a defatting/disinfecting process. In the defatting/disinfecting process, the source bone is immersed in a solution of water and ethanol. The ethanol acts to disinfect the bone by killing viruses, bacteria, fungi, and other unwanted microorganisms and pathogens. Additionally, ethanol is a useful solvent for lipids and helps to remove lipids from the source bone. The water acts as a hydrophilic carrier to facilitate deep penetration of the ethanol into the source bone. Though any concentration of ethanol in water may be used, in a preferred embodiment, at least 40% by weight of ethanol in water is used as a defatting/disinfecting solution. More preferably, about 75% by weight of ethanol in water is used as a defatting/disinfecting solution.

Bone material utilized in the fabrication of the osteoimplant also may be autogenous, allogenic, or xenogenic in origin. Autogenous bone is bone taken from the patient for whom the osteoimplant is produced. Allogenic bone is taken from another human donor. Xenogenic bone is taken from an animal donor. In a preferred embodiment of the invention, the xenogenic bone material is bovine bone material, porcine bone material, or a combination thereof. Additionally, bone material utilized in this invention may be from cortical bone, cancellous bone, corticocancellous bone, or mixtures thereof.

The bone material used in fabrication of the osteoimplant may be in any one of numerous physical forms. For example, the bone material may be produced by grinding the source bone into small particles. In another example, the bone material may be produced by milling whole bone to produce fibers, chipping whole bone, cutting whole bone, fracturing whole bone in liquid nitrogen, or otherwise disintegrating the bone tissue. In yet another example, the bone material may be produced by pulverizing, crushing, mashing, or pounding source bone. Particles may optionally be subjected to one or more sieving steps in order to separate particles of a specific size or range of sizes. It may be advantageous to select for smaller particles because smaller particles may bind more strongly to the polymer matrix, may have a greater reinforcement effect because of their larger surface area, and may have a greater toughening effect on the osteoimplant.

In a preferred embodiment of the present invention, the bone material is produced by grinding the source bone into small grains, granules, or a powder. The powdered bone material may assume any range of sizes appropriate for inclusion in the bone-polymer osteoimplant. In a preferred embodiment, the powdered bone material has a particle size of 0.1-1 mm. In another preferred embodiment, the powdered bone material has a particle size of 0.01-0.1 mm, which may be advantageous because it is thought that cells may grow better within an implant with a porosity of 0.03-0.08 mm. In another preferred embodiment, the powdered bone material has a particle size of 0.001-0.01 mm, which may be advantageous because of the increased reinforcement effect of powder within this range of sizes. Again, the powdered bone material may be sieved in order to exclude undesirable powder sizes, or may be a mixture of different sizes in order to achieve combined effects.

The bone material of the osteoimplant may be in the form of elongated rods or shards with high thickness-to-length ratios. Inclusion of elongate bone material in a bone-polymer osteoimplant results in an osteoimplant with especially good compressive strength. Elongated bone material may be obtained, for example, by milling or shaving the surface of a source bone. Employing a milling technique, one can obtain a mass of elongate bone material containing at least about 60 weight percent, preferably at least about 70 weight percent, and most preferably at least about 80 weight percent of elongate bone material. The elongate bone material may possess a median length of from about 2 to about 200 mm or more, and preferably from about 10 to about 100 mm; a median thickness of from about 0.05 to about 2 mm, and preferably from about 0.05 to about 0.1 mm; and a median width of from about 1 mm to about 20 mm, and preferably from about 0.05 to about 0.1 mm. The elongate bone material may possess a median length to median thickness ratio of at least about 50:1 up to about 500:1 or more, and preferably greater than about 50:1. The elongate bone material also may possess a median length to median width ratio of at least about 10:1 up to about 200:1 or more, and preferably greater than about 50:1.

Another procedure for obtaining elongate bone material, particularly useful for pieces of bone up to about 100 mm in length, is the bone processing mill described in U.S. Pat. No. 5,607,269, the disclosure of which is incorporated herein by reference in its entirety. Use of this bone mill results in the production of long, thin strips that quickly curl lengthwise to provide tubular-like bone particles. If desired, elongate bone material may be graded into different sizes to reduce or eliminate less desirable sizes of particles. Elongate bone material also may be cut into different sizes in accordance with the dimensions of devices in which the elongate bone material is to be used. In overall appearance, elongate bone particles can be described as filaments, fibers, threads, slender or narrow strips, etc.

In a preferred embodiment of the present invention, the bone material is subjected to surface treatment with a surface treatement agent to promote binding of the bone to other components of the osteoimplant such as the polymer and bioactive agents. The surface treatment agents may include, for example, chemically active coupling compounds, block copolymers, and random copolymers. The surface treatment agents may be chemically or physically coupled to the bone particles and other components, resulting in enhanced binding between the bone material and other components of the osteoimplant, improved mechanical integrity, and other enhanced properties.

Other treatments, for example thermal (freezing) or irradiation processes, also may be carried out for various advantageous effects upon the bone material.

Any biocompatible polymer may be used in the fabrication of the osteoimplant. The polymer acts as a rigid matrix in which the bone material is held and acts to reinforce the osteoimplant. Biocompatible polymers used in the osteoimplant may be natural, semi-synthetic, or synthetic in origin.

Natural biocompatible polymers, for example, may be chosen from the group including biological adhesives such as fibrin glue, fibrinogen, thrombin, mussel adhesive protein, silk, elastin, collagen, casein, gelatin, albumin, keratin, chitin or chitosan, natural or modified polysaccharides, polyethylene glycol derivatives, and starches.

Semi-synthetic biocompatible polymers include, but are not limited to, genetically-engineered protein and cellulose polymers, for example, silk-like protein, proNectin® (commercially available from SoloHill Engineering, Ann Arbor, Mich.), and collagen-like protein. Other examples of semi-synthetic polymers include protein and cellulose derivatives, for example, ethyl cellulose, methyl cellulose, carboxy methyl cellulose, hydroxy ethyl cellulose, sodium carboxymethylcellulose, hydroxy propyl cellulose, cellulose esters, and combinations and mixtures thereof.

Synthetic bio-compatible polymers include, for example, epoxy-based compounds, polyacrylates, polymethacrylates, fluoropolymers, silicone polymers, polyurethanes, polyesters, polyethers, polyolefins, vinyl polymers, polysulfones, polyacetals, polyimides, polyamides, polycarbonates, poly(hydroxybutyrates), polyanhydrides, poly(ortho esters), bisphenol-A based poly(phosphoesters), polyphenylenesulfides, polyamideimides, polyaryletherketones, polyarylethernitriles, aromatic polyhydroxy-ethers, polyphenyleneoxides and their combinations. Other examples of synthetic polymers include, but are not limited to, amino acid-derived polycarbonates, amino acid-derived polyarylates, polyarylates derived from dicarboxylic acids and amino acid-derived diphenols, anionic polymers derived from L-tyrosine, and polyarylate random block copolymers.

The polymer used in fabrication of the osteoimplant may likewise be a natural, synthetic or semisynthetic bioresorbable polymer that will be slowly absorbed by the body following implantation of the osteoimplant. Examples of bioresorable polymers include, but are not limited to, polylactic acid, polyglycolic acid, polylactic-co-glycolic acid, polyglycolates, polydioxanone, polycaprolactone, polycarbonates, polyorthoesters, polyamino acids, polyanhydrides, polyhydroxybutyrate, polyhydroxyvalyrate, poly(propylene glycol-co-fumaric acid), polyhydroxyalkanoates, polyphosphazenes, poly(alkylcyanoacrylates), polyarylates, amino acid-derived polymers, amino acid-based polymers, particularly tyrosine-based polymers including tyrosine-based polycarbonates. Still further non-limiting examples of bioresorbable polymers include cellulose and its derivatives and combinations thereof.

Any appropriate solvent may be used in the method for producing an osteoimplant. For a given polymer, a solvent should be chosen that adequately dissolves the polymer. Generally, solvents also should have low toxicity, be easy to remove by vaporization or extraction, and cause minimal damage to bone components. Examples of solvents include, but are not limited to, alcohols such as methanol, ethanol and isopropyl alcohol; ketones such as acetone and methyl ethyl ketone; ethers such ethyl ether and tetrahydrofuran; esters such as ethyl acetate; chloride solvents such as chloroform and methylene chloride; fluoride solvents; aliphatic and aromatic hydrocarbons such hexane, heptane, cyclohexane and benzene; and supercritical liquids such as super critical carbon dioxide.

In a preferred embodiment, the solvent is tetrahydrofuran (THF) or another similar substance, such as acetone, ethyl acetate, super critical carbon dioxide, and ethyl ether.

The amount of solvent used to produce the osteoimplant preferably is dependent upon the effect of the solvent in assisting the mixing of the bone material and polymer, the entanglment of polymeric chains, the mechanical integrity of the osteoimplant, and the biological activity of the osteoimplant. Preferably, the solvent component of the mixture used to form the osteoimplant should be from about 10% to about 90% by weight of the mixture. More preferably, the solvent should be from about 20% to about 30% by weight of the mixture.

In a preferred embodiment of the present invention, porosity generation agents may be included in the osteoimplant. Porosity generation agents may be mixed with the bone material, solvent, and polymer before the mixture is formed into the shape of the osteoimplant. Preferrably, the porosity generation agents are choosen such that they are not significantly soluble in the solvent choosen for mixing of the bone material and polymer. Otherwise, the porosity generation agents may not perform their intended function.

After the mixture is formed into the shape of an osteoimplant, the porosity generation agents may be removed from the formed osteoimplant, leaving behind voids or a porous structure in the osteoimplant. The porosity generation agents may be removed before or after the solvent is substantially removed. Methods for removing the porosity generation agents include, for example, immersing the osteoimplant in water or another solvent in which the porosity generation agents are soluble for a time sufficient to remove substantially all of the porosity generation agents. The porosity generation agents also may be removed from the molded osteoimplant through chemical decomposition. One skilled in the art will appreciate still others methods by which the porosity generation agents may be extracted or removed from the formed osteoimplant. Exemplary porosity generation agents include, but not limited to, inorganic salts such as sodium chloride and potassium chloride, and water soluble small organic molecules such as glucose and other sugars. The porosity generation agent may preferably be used in a powder form with the particle size comparable to the desired pore size.

In another preferred embodiment of the present invention, one or more biological agents or additives also may be included in the osteoimplant by mixing the agent or additive into the bone/polymer/solvent mixture or by contacting or soaking the osteoimplant with the agent or additive or its solution anytime before or after implantation to incorporate or associate the additive or agent with the osteoimplant. Examples of biological agents and additives include, but are not limited to, antibiotics, growth factors, fibrin, bone morphogenetic factors, bone growth agents, chemotherapeutics, pain killers, bisphosphonates, strontium salt, fluoride salt, magnesium salt, and sodium salt. The biological agent or additive may be in a purified form, partially purified form, recombinant form, or any other form appropriate for inclusion in the osteoimplant. It is preferred that the agent or additive be free of impurities and contaminants.

Antibiotics and antiretroviral drugs are useful for treatment of or prophylaxis for an infection. Examples of antibiotics that may be included in the osteoimplant are tetracyclines, vancomycin, cephalosporins, erythromycin, bacitracin, neomycin, penicillin, polymycin B, biomycin, chloromycetin, streptomycins, cefazolin, ampicillin, azactam, tobramycin, clindamycin, gentamicin, and aminoglycocides such as tobramycin and gentamicin.

Growth factors may be included in the osteoimplant to encourage bone or tissue growth. Non-limiting examples of growth factors that may be included are collagen, platelet derived growth factor (PDGF), transforming growth factor b (TGF-b), insulin-related growth factor-I (IGF-I), insulin-related growth factor-II (IGF-II), fibroblast growth factor (FGF), beta-2-microglobulin (BDGF II), and bone morphogenetic factors. Bone morphogenetic factors are growth factors whose activity is specific to bone tissue including, but not limited to, proteins of demineralized bone, demineralized bone matrix (DBM), and in particular bone protein (BP) or bone morphogenetic protein (BMP). Osteoinductive factors such as fibronectin (FN), osteonectin (ON), endothelial cell growth factor (ECGF), cementum attachment extracts (CAE), ketanserin, human growth hormone (HGH), animal growth hormones, epidermal growth factor (EGF), interleukin-1 (IL-1), human alpha thrombin, transforming growth factor (TGF-beta), insulin-like growth factor (IGF-1), platelet derived growth factors (PDGF), and fibroblast growth factors (FGF, bFGF, etc.) also may be included in the osteoimplant.

Still other growth agents include nucleic acid sequences that encode an amino acid sequence or an amino acid sequence itself wherein the amino acid sequence facilitates bone growth, bone healing, or tissue growth. For example, leptin is known to inhibit bone formation. Any nucleic acid or amino acid sequence that negatively impacts leptin, a leptin ortholog, or a leptin receptor may be included in the osteoimplant. As a specific example, antisense leptin nucleic acid may be included in the osteoimplant to inhibit leptin amino acid formation, thereby avoiding any inhibitory effects leptin may have on bone regeneration or growth. Other specific examples are a leptin antagonist and a leptin receptor antagonist.

Other biological agents or additives that may be included in the osteoimplant are chemotherapeutics such as cis-platinum, ifosfamide, methotrexate and doxorubicin hydrochloride. Those skilled in the art are capable of determining other chemotherapeutics that would be suitable for use in the invention.

The biological agent or additive also may be a pain killer or anti-inflammatory, such as non-steroidal anti-inflammatory drugs (NSAID). Examples of pain killers appropriate for inclusion in the osteoimplant include, but are not limited to, lidocaine hydrochloride, bipivacaine hydrochloride, ibuprofren, and NSAIDs such as ketorolac tromethamine.

Another biological agent or additive that may be included in the osteoimplant is a bisphosphonate. Examples of bisphosphonates are alendronate, clodronate, etidronate, ibandronate, (3-amino-1-hydroxypropylidene)-1,1-bisphosphonate (APD), dichloromethylene bisphosphonate, aminobisphosphonatezolendronate and pamidronate.

Still other examples of biological agents and additives that may be included in the osteoimplant are biocidal/biostatic sugars such as dextran and glucose; peptides; vitamins; inorganic elements; co-factors for protein synthesis; hormones; endocrine tissue or tissue fragments; synthesizers; enzymes such as collagenase, peptidases, and oxidases; polymer cell scaffolds with parenchymal cells; angiogenic agents; antigenic agents; cytoskeletal agents; cartilage fragments; living cells such as chondrocytes, bone marrow cells, mesenchymal stem cells, natural extracts, genetically engineered living cells, or otherwise modified living cells; autogenous tissues such as blood, serum, soft tissue, and bone marrow; bioadhesives; periodontal ligament chemotactic factor (PDLGF); somatotropin; bone digestors; antitumor agents; immuno-suppressants; and permeation enhancers such as fatty acid esters including laureate, myristate, and stearate monoesters of polyethylene glycol.

In another preferred embodiment of the invention, a reinforcing component or structure such as a fiber, fibrous web, woven textile, nonwoven textile, mesh, xerogel, nonflexible structural member or semiflexible structure member made from a natural, synthetic, or semisynthetic material, or combinations thereof may be added to the osteoimplant. The reinforcing component may be useful to strengthen the osteoimplant. Materials suitable for constructing the reinforcing component include, but are not limited to, collagen, tendons, keratin, cellulosics, ceramics, synthetic polymers, glass, metals and metal alloys, and calcium phosphates. The reinforcing component may be nonbioresorbable but preferably is bioresorbable in order to facilitate the remodeling process. Where practicable, it generally is advantageous to orient the reinforcing component or structure substantially parallel to the axis of the forces that can be expected to be exerted against the osteoimplant following its installation in the body.

The bone material, polymer, and solvent preferably are mixed for a suitable time to ensure even distribution. It is preferred that the components be agitated to provide a substantially uniform distribution of bone, polymer, and other additives. The bone/polymer/solvent mixture then may be molded into the desired osteoimplant shape. Osteoimplant shapes include, but are not limited to, a sheet, plate, dish, cone, pin, screw, tube, tooth, tooth root, bone, bone portion, wedge, wedge portion, cylinder, dowel, intervertebral implant, and suture anchor. Examples of osteoimplants shaped to replace entire bones include, but are not limited to, the ethmoid, frontal, nasal, occipital, parietal, temporal, mandible, maxilla, zygomatic, cervical vertebra, thoracic vertebra, lumbar vertebra, sacrum, rib, sternum, clavicle, scapula, humerus, radius, ulna, carpal bones, metacarpal bones, phalanges, ilium, ischium, pubis, femur, tibia, fibula, patella, calcaneus, tarsal, and metatarsal bones. After forming the implant, the implant may undergo further processing such as machining, sanding, shaving, or coating with suitable materials.

The molding operation may be executed in any one of numerous fashions including, but not limited to, press-molding and injection molding. Additional methods for shaping the osteoimplant include any of the various extrusion procedures, rolling, solvent-casting, gel-casting, cast-molding, vacuum-forming, sintering, melt-forming, blow-molding, leach molding (where an additional phase is removed by solvent after formation), and leavening (where a gas is formed by decomposition of the additional phase).

For some molding methods, for example press molding and injection molding, the mixture may preferrably be held under a molding force for a time sufficient to allow the polymer to adequately weld and bind to the bone material and other components. This may be desirable so that the polymer chains can entangle with each other and also form effective interactions with other components of the osteoimplant. Additionally, the mixture may preferrably be held under a molding force sufficient to effect the formation of a desirable polymer matrix and strong binding between the osteoimplant components. One skilled in the art will appreciate that the amount of solvent in the mixture used to form the osteoimplant, the molding force, and the holding time are interrelated. Greater amounts of solvent, for example, may result in a softer mixture that might require less molding force and shorter holding time. However, because more solvent may need to be removed from the formed osteoimplant, the osteoimplant might experience undesired deformation upon drying.

The solvent may be substantially removed from the molded osteoimplant by any one of numerous processes. In a preferred embodiment, the solvent is substantially removed by freeze drying the molded implant. Alternatively, the solvent may be substantially removed by simply air drying the implant, placing the implant under vacuum, heating the implant, or extracting the solvent with another solvent that is easier to remove. In a preferred embodiment, the solvent may be removed in a nitrogen atmosphere. A person having ordinary skill in the art will appreciate the many different methods by which the solvent may be substantially removed from the osteoimplant, using the guidelines provided herein. Substantial removal of the solvent from the osteoimplant may result in the formation of voids and passageways in the osteoimplant. These voids or passageways may be osteoconductive because they provide a matrix wherein new bone cells are supported. These voids or passageways also may serve as channels or reservoirs for any of the additives or agents mentioned previously.

Solvents may be easier to remove from the osteoimplant than are aqueous solutions and wetting agents. Substantial removal of aqueous solutions and wetting agents from the osteoimplant may require drying or evaporation at elevated temperature. Solvents, however, may be substantially removed from the osteoimplant at lower temperatures, preferably at or about room temperature. Lower temperatures during removal of the solvent may be desirable because higher temperatures may damage the biological activity of any of the biological components that might be present in the osteoimplant (e.g., proteins, DBM, etc.). Therefore, the use of solvents in processing the bone material and polymer may result in an osteoimplant with increased biological activity compared to an osteoimplant processeed using aqueous solutions and wetting agents. In a preferred embodiment, the solvent is substantially removed from the osteoimplant at room temperature so as to avoid undue damage to the biological activity of the osteoimplant.

The osteoimplant may be installed at any hard tissue repair site. Such repair sites may result from, for example, injury, defects created during surgery, infection, malignancy, or developmental malformation. The osteoimplant may be utilized in a wide variety of orthopedic, periodontal, neurosurgical, and oral and maxillofacial surgical procedures. These procedures include, for example, the repair of simple and compound fractures and non-unions, external and internal fixations, joint reconstructions such as arthrodesis, general arthroplasty, cup arthroplasty of the hip, femoral and humeral head replacement, femoral head surface replacement and total joint replacement, repairs of the vertebral column including spinal fusion and internal fixation, tumor surgery such as deficit filling, discectomy, laminectomy, excision of spinal cord tumors, anterior cervical and thoracic operations, repairs of spinal injuries, scoliosis, lordosis and kyphosis treatments, intermaxillary fixation of fractures, mentoplasty, temporomandibular joint replacement, alveolar ridge augmentation and reconstruction, onlay bone grafts, implant placement and revision, and sinus lifts. The osteoimplant can be implanted at the bone repair site using any suitable affixation means, for example, sutures, staples, bioadhesives, and the like.

In another exemplary embodiment, the invention provides an osteoimplant created by the previous method.

The invention now will be described in more detail with reference to the following non-limiting examples.

### EXAMPLES

Poly (D,L-lactic acid co L-lactic acid, 30/70) (PLA) (Resomer® LR 708, commercially available from Beohringer-Ingelheim Corp., GmbH, Germany) was mixed with anhydrous tetrahydrofuran (THF) (commercially available from Sigma-Aldrich, Milwaukee, Wis.) at a ratio of 20 parts PLA to 80 parts THF. PLA pellets agglomerated upon being mixed with THF. The agglomerates were broken periodically until the polymer was completely dissolved in THF to form a visually gel-like solution. The solution was stored in a dryer.

Bovine bone samples were ground into powder with particle sizes in a range from 250 to 850 microns. The procedure used to make these bone powder samples is as follows:
1. Clean soft tissue from bones with scalpel and gauze.
2. Saw cortical bone into 1 cm rings and cut the rings into thirds.
3. Wash bone pieces in a sufficient quantity of sterile water for 15 minutes.
4. Soak bone pieces in a sufficient quantity of 70% ethanol for 15 minutes.
5. Freeze dry bone pieces for a minimum of 2 to 3 days, larger pieces requiring longer freeze dry cycles.
6. Grind bone and sieve to 250 to 850 microns.

The demineralized bone matrix (DBM) samples were made by treating the bone powder with hydrochloric acid (HCl) solution to dissolve the mineral components. The procedure is as follows:
1. Demineralize the regular bone powder in 0.025% v/v Triton X-100 in 0.6N HCl at room temperature for 30 minutes using stir plate and stir bar (15 mL/g mineralized bone). Decant off liquid.
2. Demineralize in 0.6N HCl for 2 hours, using stir plate and stir bar (15 mL/g mineralized bone). Decant off liquid.
3. Wash bone particles in sterile water until solution pH>4 (15 mL/g mineralized bone).
4. Stir particles on stir plate in 70% ethanol for 1 hour at room temperature (7 mL/g mineralized bone). Decant off liquid.
5. Stir particles on stir plate in fresh water for 5 minutes (15 mL/g mineralized bone). Decant off liquid.
6. Place bone particles in sterilized containers and freeze dry as normal.

Bone powder and DBM were measured based on pre-determined ratios and mixed with a pre-determined amount of PLA solution. The mixtures were further mixed by stretching-folding for at least 10 times. Then, the samples were broken into small pieces and placed in flowing air to vaporize the THF until the solvent content inside the mixture was reduced to about 20% by weight based on the total weight of the sample. The partially dried mixtures were stored in sealed bottles to wait for molding. The ratio of bone to DBM used in the present example was in a range from 100:0 to 50:50 in weight. The total percentage of regular bone and DBM in samples was in a range from 60% to 90% by weight.

The partially dried samples were loaded into a mold that had a cylindrical cavity with a rod aligning in the center. The diameter of the cylinder and the central hole was 10 mm and 3 mm, respectively. The length of the samples was 10 mm to 30 mm. The materials inside the mold were manually pressed at approximately 10 to 100 MPa for 10 seconds to 1 minute. Then, the mold with the sample inside was held with a clamp for about 1 to 3 hours to wait for polymer chain entanglement so that a continuous and well-welded polymer matrix formed. After the samples were released from the mold, they were placed under a hood to dry in air until the reduction in sample weight due to vaporization of the solvent was less than 1 mg per gram of sample per day.

The cylinders were tested under static axial compression. Specimens were rehydrated for 1 minute, blotted dry, and placed in between parallel stainless steel platens. Compression was performed in displacement control mode at a rate of 1 inch per minute. Compression continued until specimens reached structural failure. Stress and strain values were calculated for each test and plotted for analysis.

Table 1 lists the maximum strength and modulus for different sample compositions.

TABLE 1

| | | | | Ultimate | |
|---|---|---|---|---|---|
| | PLA | Bone | DBM | Compression | |
| Sample | (wt-%) | (wt-%) | (wt-%) | Strength (MPa) | Modulus (MPa) |
| A | 10 | 45.0 | 45.0 | 39 | 327 |
| B | 10 | 80.7 | 9.2 | 44 | 647 |
| C | 11 | 89.0 | 0.0 | 54 | 790 |
| D | 20 | 50.0 | 30.0 | 45 | 401 |
| E | 20 | 80.0 | 0.0 | 42 | 902 |
| F | 20 | 72.5 | 7.3 | 41 | 838 |
| G | 30 | 62.7 | 7.5 | 42 | 616 |
| H | 30 | 40.0 | 30.0 | 42 | 505 |
| I | 30 | 70.0 | 0.0 | 42 | 689 |
| J | 39 | 54.9 | 5.7 | 34 | 244 |
| K | 39 | 60.6 | 0.0 | 30 | 284 |
| L | 40 | 30.2 | 29.8 | 46 | 596 |

Samples A (PLA:Bone:DBM ratio of 10:45:45) and L (PLA:Bone:DBM ratio of 40:30:30) were soaked in water for period of 122 and 146 hours. Water absorption and dimension change of the samples were measured and are listed in Table 2.

TABLE 2

| Soaking | | | Change | | Change |
|---|---|---|---|---|---|
| time (hr) | | Sample A | (%) | Sample L | (%) |
| 0 | Length | 10.91 | 0 | 13.92 | 0 |
| | Diameter | 10.05 | 0 | 9.55 | 0 |
| 122 | Length | 12.53 | 14.8 | 14.2 | 2.0 |
| | Diameter | 10.92 | 8.7 | 9.76 | 2.2 |
| 146 | Length | 12.56 | 15.1 | 14.27 | 2.5 |
| | Diameter | 10.9 | 8.5 | 9.875 | 3.4 |

The invention has been described with reference to particularly preferred embodiments and examples. Those skilled in the art will appreciate that various modifications may be made to the invention without departing from the spirit and scope thereof.

What is claimed is:

1. A method for producing an osteoimplant device comprising:
mixing bone material, a solvent, and a polymer that is substantially soluble in the solvent;
forming the mixture into the shape of an osteoimplant; and
removing substantially all of the solvent from the osteoimplant.

2. The method of claim 1, wherein the bone material is selected from the group consisting of autogenic, allogenic, and xenogenic source bone, and combinations and mixtures thereof.

3. The method of claim 1, wherein the bone material is selected from the group consisting of bone powder; bone granules; bone chips; bone shards; bone flakes; products of ground, pulverized, and chipped bone; and combinations and mixtures thereof.

4. The method of claim 1, wherein the bone material comprises nondemineralized source bone, fully demineralized source bone, partially demineralized source bone, and combinations and mixtures thereof.

5. The method of claim 1, wherein the solvent is selected from the group consisting of alcohols, ketones, ethers, esters, chloride solvents, fluoride solvents, aliphatic hydrocarbons, aromatic hydrocarbons, supercritical liquids, and combinations and mixtures thereof.

6. The method of claim 5, wherein the solvent is tetrahydrofuran.

7. The method of claim 1, wherein the polymer is a natural, synthetic, or semi-synthetic polymer.

8. The method of claim 1, wherein the polymer is substantially bioresorbable.

9. The method of claim 7, wherein the natural polymer is selected from the group consisting of fibrin glue, fibrinogen, thrombin, mussel adhesive protein, silk, elastin, collagen, casein, gelatin, albumin, keratin, chitin or chitosan, natural or modified polysaccharides, starches, and combinations and mixtures thereof.

10. The method of claim 7, wherein the synthetic polymer is selected from the group consisting of epoxy-based compounds, polyacrylates, polymethacrylates, fluoropolymers, silicone polymers, polyurethanes, polyesters, polyethers, polyolefins, vinyl polymers, polysulfones, polyacetals, polyimides, polyamides, polycarbonates, bisphenol-A based poly(phosphoesters), polyphenylenesulfides, polyamideimides, polyaryletherketones, polyarylethernitriles, aromatic polyhydroxy-ethers, polyphenyleneoxides, amino acid-derived polycarbonates, amino acid-derived polyarylates, polyarylates derived from dicarboxylic acids and amino acid-derived diphenols, anionic polymers derived from L-tyrosine, polyarylate random block copolymers, and combinations and mixtures thereof.

11. The method of claim 7, wherein the semi-synthetic polymer is selected from the group consisting of genetically-engineered protein polymers, ethyl cellulose, methyl cellulose, carboxy methyl cellulose, hydroxy ethyl cellulose, sodium carboxymethylcellulose, hydroxy propyl cellulose, cellulose esters, and combinations and mixtures thereof.

12. The method of claim 8, wherein the bioresorbable polymer is selected from the group consisting of polylactic acid, polyglycolic acid, polylactic-co-glycolic acid, polyglycolates, polydioxanone, polycaprolactone, polycarbonates, polyorthoesters, polyamino acids, polyanhydrides, polyhydroxybutyrate, polyhydroxyvalyrate, poly(propylene glycol-co-fumaric acid), polyhydroxyalkanoates, polyphosphazenes, poly(alkylcyanoacrylates), polyarylates, amino acid-based polymers, cellulose, celluose derivatives, and combinations and mixtures thereof.

13. The method of claim 1, wherein substantially all of the solvent is removed from the osteoimplant by freeze drying.

14. The method of claim 1, wherein substantially all of the solvent is removed from the osteoimplant by placing the implant under vacuum.

15. The method of claim 1, wherein substantially all of the solvent is removed from the osteoimplant by heating the implant.

16. The method of claim 1, wherein substantially all of the solvent is removed from the osteoimplant by drying the osteoimplant in circulating air at room temperature.

17. The method of claim 1, further comprising adding a biological agent or additive to the osteoimplant.

18. The method of claim 17, wherein the biological agent or additive is selected from the group consisting of antibiotics, growth factors, fibrin, bone morphogenetic factors, bone growth agents, chemotherapeutics, pain killers, bisphosphonates, strontium salt, fluoride salt, magnesium salt, sodium salt, and combinations and mixtures thereof.

19. The method of claim 1, further comprising adding a reinforcing component to the osteoimplant.

20. The method of claim 19, wherein the reinforcing component is selected from the group consisting of fibers, fibrous webs, woven textiles, nonwoven textiles, mesh, and combinations and mixtures thereof.

21. The method of claim 1, wherein mixing additionally comprises mixing a porosity generation agent.

22. The method of claim 21, wherein the porosity generation agent is selected from the group consisting of sodium chloride, potassium chloride, other inorganic salts, glucose, sugars, other small molecular weight organic compounds, and combinations and mixtures thereof.

23. The method of claim 1, wherein the bone material is treated with a surface treatment agent.

24. The method of claim 23, wherein the surface treatment agent chemically or physically binds to the bone material and polymer.

25. The method of claim 1, wherein forming the mixture into the shape of an osteoimplant comprises a procedure selected from the group consisting of press-molding, injection molding, extrusion, rolling, solvent-casting, gel-casting, cast-molding, vacuum-forming, sintering, melt-forming, blow-molding, leach molding, leavening, and combinations thereof.

26. An osteoimplant device produced by the method of claim 1.

27. The device of claim 26, wherein the osteoimplant is in the form of one or more selected from the group consisting of a sheet, plate, dish, cone, pin, screw, tube, tooth, tooth root, bone, bone portion, wedge, wedge portion, cylinder, dowel, intervertebral implant, suture anchor, and combinations thereof.

28. The device of claim 26, wherein the bone material is selected from the group consisting of autogenic, allogenic, and xenogenic source bone, and combinations and mixtures thereof.

29. The device of claim 26, wherein the bone material is selected from the group consisting of bone powder; bone granules; bone chips; bone shards; bone flakes; products of ground, pulverized, and chipped bone; and combinations and mixtures thereof.

30. The device of claim 26, wherein the bone material comprises nondemineralized source bone, fully demineralized source bone, partially demineralized source bone, and combinations and mixtures thereof.

31. The device of claim 26, wherein the solvent is selected from the group consisting of alcohols, ketones, ethers, esters, chloride solvents, fluoride solvents, aliphatic hydrocarbons, aromatic hydrocarbons, supercritical liquids, and combinations and mixtures thereof.

32. The device of claim 31, wherein the solvent is tetrahydrofuran.

33. The device of claim 26, wherein the polymer is a natural, synthetic, or semi-synthetic polymer.

34. The device of claim 26, wherein the polymer is substantially bioresorbable.

35. The device of claim 33, wherein the natural polymer is selected from the group consisting of fibrin glue, fibrinogen, thrombin, mussel adhesive protein, silk, elastin, collagen, casein, gelatin, albumin, keratin, chitin or chitosan, natural or modified polysaccharides, starches, and combinations and mixtures thereof.

36. The device of claim 33, wherein the synthetic polymer is selected from the group consisting of epoxy-based compounds, polyacrylates, polymethacrylates, fluoropolymers, silicone polymers, polyurethanes, polyesters, polyethers, polyolefins, vinyl polymers, polysulfones, polyacetals, polyimides, polyamides, polycarbonates, bisphenol-A based poly(phosphoesters), polyphenylenesulfides, polyamideimides, polyaryletherketones, polyarylethernitriles, aromatic polyhydroxy-ethers, polyphenyleneoxides, amino acid-derived polycarbonates, amino acid-derived polyarylates, polyarylates derived from dicarboxylic acids and amino acid-derived diphenols, anionic polymers derived from L-tyrosine, polyarylate random block copolymers, and combinations and mixtures thereof.

37. The device of claim 33, wherein the semi-synthetic polymer is selected from the group consisting of genetically-engineered protein polymers, ethyl cellulose, methyl cellulose, carboxy methyl cellulose, hydroxy ethyl cellulose, sodium carboxymethylcellulose, hydroxy propyl cellulose, cellulose esters, and combinations and mixtures thereof.

38. The device of claim 34, wherein the bioresorbable polymer is selected from the group consisting of polylactic acid, polyglycolic acid, polylactic-co-glycolic acid, polyglycolates, polydioxanone, polycaprolactone, polycarbonates, polyorthoesters, polyamino acids, polyanhydrides, polyhydroxybutyrate, polyhydroxyvalyrate, poly(propylene glycol-co-fumaric acid), polyhydroxyalkanoates, polyphosphazenes, poly(alkylcyanoacrylates), polyarylates, amino acid-based polymers, cellulose, celluose derivatives, and combinations and mixtures thereof.

39. The device of claim 26, wherein substantially all of the solvent is removed from the osteoimplant by freeze drying.

40. The device of claim 26, wherein substantially all of the solvent is removed from the osteoimplant by placing the implant under vacuum.

41. The device of claim 26, wherein substantially all of the solvent is removed from the osteoimplant by heating the implant.

42. The device of claim 26, wherein substantially all of the solvent is removed from the osteoimplant by drying the osteoimplant in circulating air at room temperature.

43. The device of claim 26, further comprising adding a biological agent or additive to the osteoimplant.

44. The device of claim 43, wherein the biological agent or additive is selected from the group consisting of antibiotics, growth factors, fibrin, bone morphogenetic factors, bone growth agents, chemotherapeutics, pain killers, bisphosphonates, strontium salt, fluoride salt, magnesium salt, sodium salt, and combinations and mixtures thereof.

45. The device of claim 26, further comprising adding a reinforcing component to the osteoimplant.

46. The device of claim 45, wherein the reinforcing component is selected from the group consisting of fibers, fibrous webs, woven textiles, nonwoven textiles, mesh, and combinations and mixtures thereof.

47. The device of claim 26, wherein mixing additionally comprises mixing a porosity generation agent.

48. The device of claim 47, wherein the porosity generation agent is selected from the group consisting of sodium chloride, potassium chloride, other inorganic salts, glucose, sugars, other small molecular weight organic compounds, and combinations and mixtures thereof.

49. The device of claim 26, wherein the bone material is treated with a surface treatment agent.

50. The device of claim 49, wherein the surface treatment agent chemically or physically binds to the bone material and polymer.

51. The device of claim 26, wherein forming the mixture into the shape of an osteoimplant comprises a procedure selected from the group consisting of press-molding, injection molding, extrusion, rolling, solvent-casting, gel-casting, cast-molding, vacuum-forming, sintering, melt-forming, blow-molding, leach molding, leavening, and combinations thereof.
